(19) United States
(12) Patent Application Publication
(10) Pub. No.: US 2024/0082198 A1
(43) Pub. Date: Mar. 14, 2024

(54) STABILIZED ALKYL NITRITE COMPOSITIONS

(71) Applicant: EMERGENT PRODUCT DEVELOPMENT GAITHERSBURG INC., Gaithersburg, MD (US)

(72) Inventors: Daniel Cope HILL, Gaithersburg, MD (US); Anthony M. TRESTON, Gaithersburg, MD (US)

(73) Assignee: EMERGENT PRODUCT DEVELOPMENT GAITHERSBURG INC., Gaithersburg, MD (US)

(21) Appl. No.: 18/246,982

(22) PCT Filed: Sep. 30, 2021
(86) PCT No.: PCT/US2021/052921

§ 371 Date: Mar. 28, 2023

Related U.S. Application Data
(60) Provisional application No. 63/198,188, filed on Oct. 1, 2020.

(51) Int. Cl. A61K 31/21; A61K 31/122

(52) U.S. Cl. CPC A61K 31/21; A61K 31/122

(57) ABSTRACT

This disclosure describes a composition comprising an alkyl nitrite, such as isoamyl nitrite, and an effective amount of at least one stabilizing compound, such as one or more of vitamins K1, K2, and K3.

### STATEMENT OF GOVERNMENTAL INTEREST

This invention was made with government support under contract number HHSO100201700001C awarded by the Biomedical Advanced Research and Development Authority (BARDA). The government has certain rights in this invention.

### BACKGROUND

Alkyl nitrites, including isoamyl nitrite, are used to relax spasms in arteries, to control convulsions, and for relieving asthmatic paroxysm. Alkyl nitrites, such as isoamyl nitrite are also known to be useful for treating cyanide poisoning.

Despite their utility, alkyl nitrites are known to degrade quickly upon storage for extended periods of time, even under the best of conditions. Problems with degradation are especially acute when the nitrite is stored for prolonged periods of time as a considerable amount of NO gas can be produced. When stored in ampules, this NO off-gassing can cause the ampules to explode spontaneously. The degradation of alkyl nitrites is further exacerbated by heat, which can lead to violent decomposition of the alkyl nitrite and/or a fire. In view these known issues, there is a need for stable alkyl nitrite compositions that are less susceptible to degradation upon storage for extended periods of time, or upon exposure to heat.

### BRIEF SUMMARY OF THE DISCLOSURE

The present disclosure is directed to compositions comprising a mixture of a compound according to Formula I and an alkyl nitrite. More specifically, the present disclosure provides a composition comprising an alkyl nitrite and an effective amount of at least one compound of Formula I:

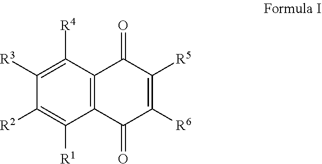

wherein:
R¹, R², R³, and R⁴ are each independently selected from the group consisting of —H, —OH, —NH₂, —SH, halogen, optionally substituted C₁-C₁₂ alkyl, optionally substituted C₁-C₁₂ alkoxy, C₁-C₄ alkylthio, —C≡N, —C(═O)H, —C(═O)OH, —C(═O)O(C₁-C₃ alkyl), and —C(═O)(C₁-C₃ alkyl); further wherein
R⁵ and R⁶ are each independently selected from the group consisting of —H, —OH, —SH, —NH₂, halogen, optionally substituted C₁-C₁₂ alkyl, optionally substituted C₂-C₇₅ alkenyl, C₁-C₄ alkylthio, —C≡N, C₁-C₁₂ alkoxy, —C(═O)H, and —C(═O)(C₁-C₃-alkyl).

In certain embodiments, the alkyl nitrite is a (C₁-C₇ alkyl nitrite.

In further embodiments, the (C₁-C₇ alkyl nitrite is selected from the group consisting of propyl nitrite, isopropyl nitrite, butyl nitrite, sec-butyl nitrite, isobutyl nitrite, tent-butyl nitrite, amyl nitrite, isoamyl nitrite, and neo-pentyl nitrite.

In some embodiments, the alkyl nitrite is isoamyl nitrite.

In some embodiments, R¹, R², R³, and R⁴ are each —H.

In some embodiments, R⁵ is an optionally substituted C₁-C₁₂ alkyl and R⁶ is an optionally substituted C₂-C₇₅ alkenyl or —H.

In some embodiments, the optionally substituted C₁-C₁₂ alkyl is selected from the group consisting of methyl, ethyl, propyl, isopropyl, butyl, sec-butyl, isobutyl, tent-butyl, pentyl, isopentyl, neo-pentyl, (1,1-dimethyl)-prop-1-yl, hexyl, heptyl, octyl, isooctyl, nonyl, decyl, cyclopentyl, cyclohexyl, and cycloheptyl.

In some embodiments, the optionally substituted C₁-C₁₂ alkyl is methyl.

In some embodiments, the optionally substituted C₂-C₇₅ alkenyl is selected from the group consisting of ethenyl, propenyl, isopropenyl, butenyl, pentenyl, hexenyl, heptenyl, (E,7R,11R)-3,7,11,15-tetramethylhexadec-2-en-1-yl, (2E)-3,7-dimethylocta-2,6-dien-1-yl, (2E,6E)-3,7,11-trimethyldodeca-2,6,10-trien-1-yl, (2E,6E,10E)-3,7,11,15-tetramethylhexadeca-2,6,10,14-tetraen-1-yl, (2Z,6E,10E)-3,7,11,15-tetramethylhexadeca-2,6,10,14-tetraen-1-yl, (2E,6E,10E,14E)-3,7,11,15,19-pentamethylicosa-2,6,10,14,18-pentaen-1-yl, (2E,6E,10E,14E,18E)-3,7,11,15,19,23-hexamethyltetracosa-2,6,10,14,18,22-hexaen-1-yl, (2Z,6E,10E,14E,18E,22E)-3,7,11,15,19,23,27-heptamethyloctacosa-2,6,10,14,18,22,26-heptaen-1-yl, (2E,6Z,10E,14E,18E,22E)-3,7,11,15,19,23,27-heptamethyloctacosa-2,6,10,14,18,22,26-heptaen-1-yl, (2E,6E,10E,14E,18E,22E)-3,7,11,15,19,23,27-heptamethyloctacosa-2,6,10,14,18,22,26-heptaen-1-yl, (2E,6E,10E,14E,18E,22E,26E)-3,7,11,15,19,23,27,31-octamethyldotriaconta-2,6,10,14,18,22,26,30-octaen-1-yl, (2E,6E,10E,14E,18E,22E,26E)-3,7,11,15,19,23,27,31-octamethyldotriaconta-2,6,10,14,18,22,26,30-octaen-1-yl, (2E,6E,10E,14E,18E,22E,26E,30E)-3,7,11,15,19,23,27,31,35-nonamethylhexatriaconta-2,6,10,14,18,22,26,30,34-nonaen-1-yl, (2E,6E,10E,14E,18E,22E,26E,30E,34E)-3,7,11,15,19,23,27,31,35,39-decamethyltetraconta-2,6,10,14,18,22,26,30,34,38-decaen-1-yl, (2E,6E,10E,14E,18E,22E,26E,30E,34E,38E)-3,7,11,15,19,23,27,31,35,39,43-undecamethyltetratetraconta-2,6,10,14,18,22,26,30,34,38,42-undecaen-1-yl, (2E,6E,10E,14E,18E,22E,26E,30E,34E,38E,42E)-3,7,11,15,19,23,27,31,35,39,43,47-dodecamethyloctatetraconta-2,6,10,14,18,22,26,30,34,38,42,46-dodecaen-1-yl, (2E,6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl, (2E,6E,10E,14E,18E,22E,26E,30E,34E,38E,42E,46E,50E)-3,7,11,15,19,23,27,31,35,39,43,47,51,55-tetradecamethylhexapentaconta-2,6,10,14,18,22,26,30,34,38,42,46,50,54-tetradecaen-1-yl, and (2E,6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl.

In some embodiments, the optionally substituted C₂-C₇₅ alkenyl is (E,7R,11R)-3,7,11,15-tetramethylhexadec-2-en-1-yl.

In some embodiments, R¹, R², R³, and R⁴ are each —H, R⁵ is methyl, and R⁶ is (E,7R,11R)-3,7,11,15-tetramethylhexadec-2-en-1-yl.

In some embodiments, the effective amount of the at least one compound of Formula I ranges from about 0.1% by weight to about 20% by weight of the composition.

In some embodiments, the effective amount of the at least one compound of Formula I ranges from about 1% by weight to about 10% by weight of the composition.

In some embodiments, the effective amount of the at least one compound of Formula I is about 10% by weight of the composition.

In some embodiments, R¹, R², R³, and R⁴ are each —H, R⁵ is methyl, and R⁶ is (E,7R,11R)-3,7,11,15-tetramethylhexadec-2-en-1-yl.

In some embodiments, the alkyl nitrite composition is packaged in an ampule under an inert atmosphere.

In some embodiments, the composition comprises an alkyl nitrite and an effective amount of a vitamin selected from the group consisting of vitamin K1, vitamin K2, vitamin K3, and combinations thereof.

In some embodiments, the vitamin is vitamin K1.

In some embodiments, the vitamin is vitamin K2.

In some embodiments, the vitamin is vitamin K3.

In some embodiments, the vitamin is menadione.

In some embodiments, the vitamin is menaquinone.

In some embodiments, the effective amount of the vitamin ranges from about 0.1% by weight to about 20% by weight of the composition.

In some embodiments, the effective amount of the vitamin ranges from about 1% by weight to about 10% by weight of the composition.

In some embodiments, the effective amount of the vitamin is about 10% by weight of the composition.

In some embodiments, the alkyl nitrite is a (C₁-C₇ alkyl nitrite.

In some embodiments, the (C₁-C₇ alkyl nitrite is selected from the group consisting of propyl nitrite, isopropyl nitrite, butyl nitrite, sec-butyl nitrite, isobutyl nitrite, tent-butyl nitrite, amyl nitrite, isoamyl nitrite, and neo-pentyl nitrite.

In some embodiments, the alkyl nitrite is isoamyl nitrite.

In some embodiments, the alkyl nitrite has a purity after storage at 40° C. for six months, ranging from about 90% to about 98%, as measured by gas chromatography (GC).

In some embodiments, the alkyl nitrite is isoamyl nitrite.

In some embodiments, the compound of Formula I is vitamin K1.

In some embodiments, the isoamyl nitrite has a purity after storage at 40° C. for six months of about 96%, as measured by GC.

### BRIEF DESCRIPTION OF THE FIGURES

The accompanying figures, which are incorporated herein, form part of the specification and illustrate embodiments of the present disclosure. Together with the description, the figures further serve to explain the principles of and to enable a person skilled in the relevant art(s) to make and use the disclosed embodiments. These figures are intended to be illustrative, not limiting.

FIG. 1 is a graph showing the stability of isoamyl nitrite over time in the presence of various concentrations of vitamin K1.

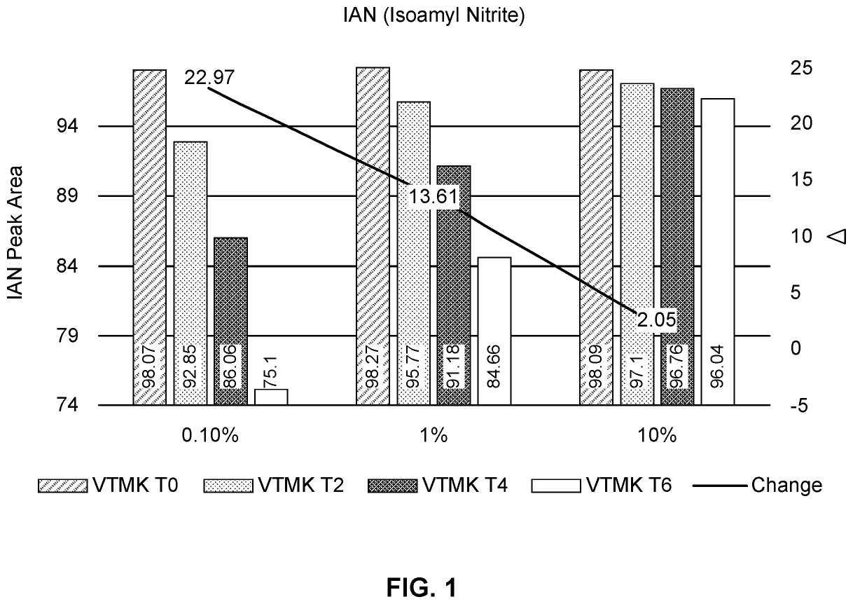

FIG. 2 is a graph showing the percentage of total impurities produced by the decomposition of isoamyl nitrite over time in the presence of various concentrations of vitamin K1.

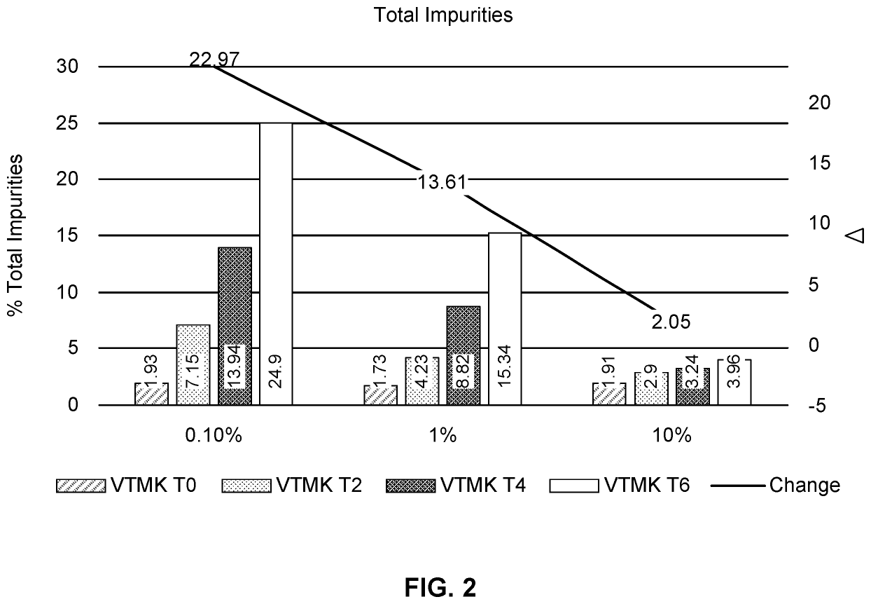

FIG. 3 is a graph showing the percentage of isoamyl alcohol produced by the decomposition of isoamyl nitrite over time in the presence of various concentrations of vitamin K1.

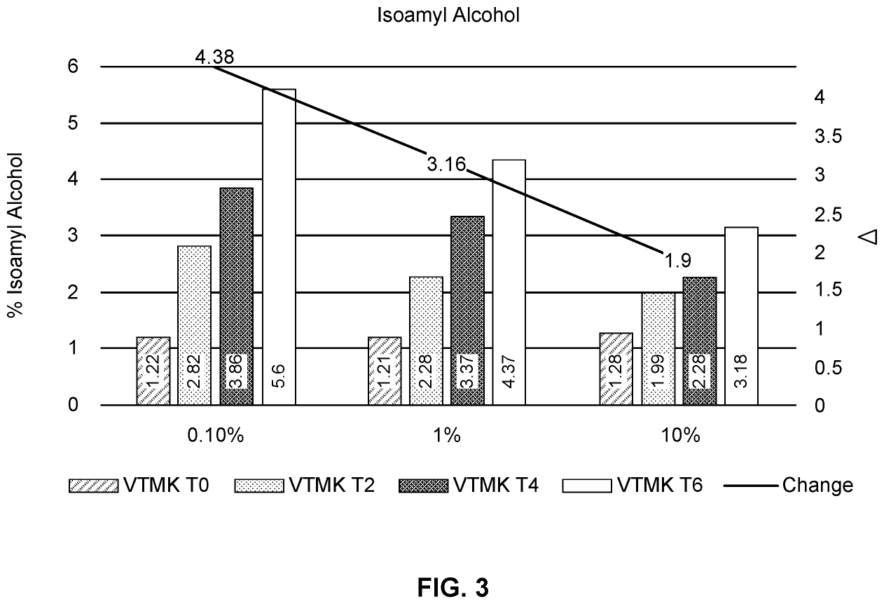

FIG. 4 is a graph showing the percentage of isovalerate produced by the decomposition of isoamyl nitrite over time in the presence of various concentrations of vitamin K1.

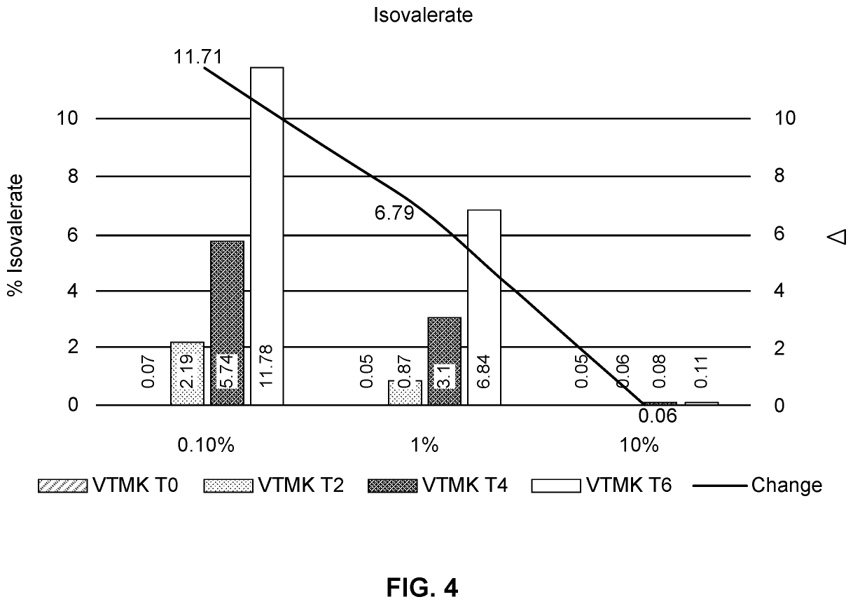

FIG. 5 is a graph showing the stability of isoamyl nitrite over time in the presence of various concentrations of epoxidized linseed oil (“ELSO”).

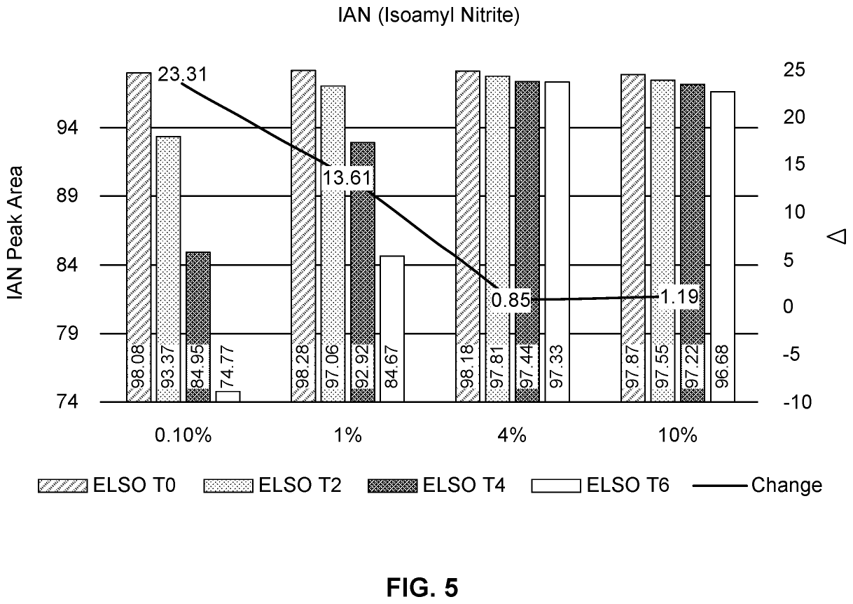

FIG. 6 is a graph showing the percentage of total impurities produced by the decomposition of isoamyl nitrite over time in the presence of various concentrations of ELSO.

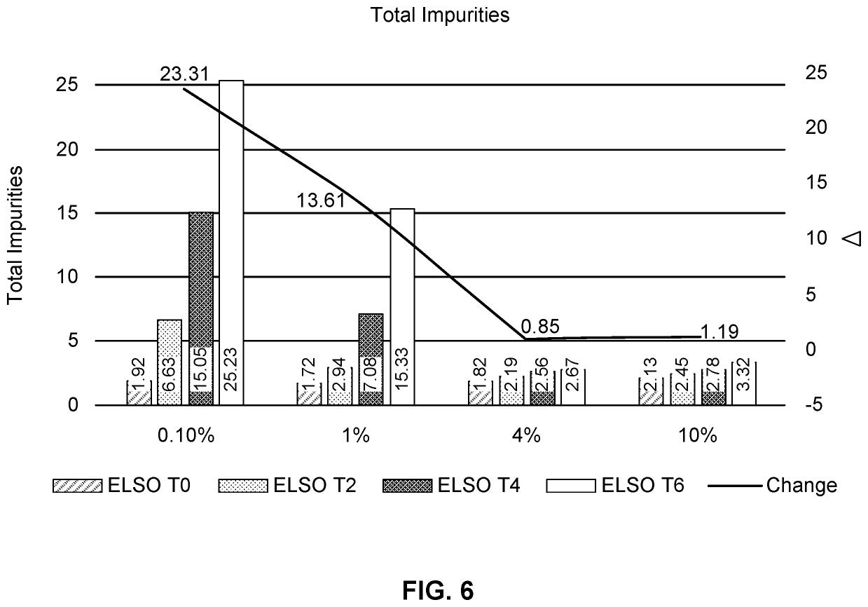

FIG. 7 is a graph showing the percentage of isoamyl alcohol produced by the decomposition of isoamyl nitrite over time in the presence of various concentrations of ELSO.

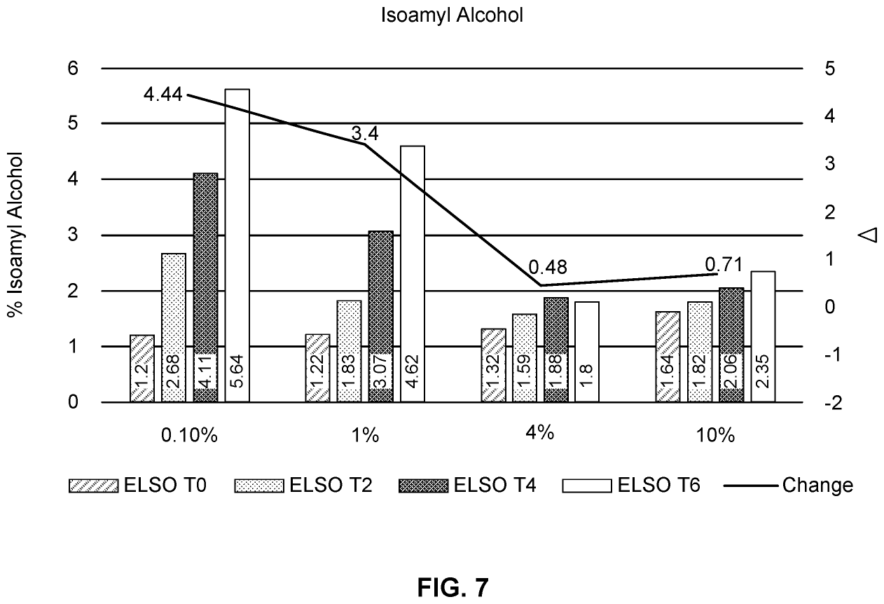

FIG. 8 is a graph showing the percentage of isovalerate produced by the decomposition of isoamyl nitrite over time in the presence of various concentrations of ELSO.

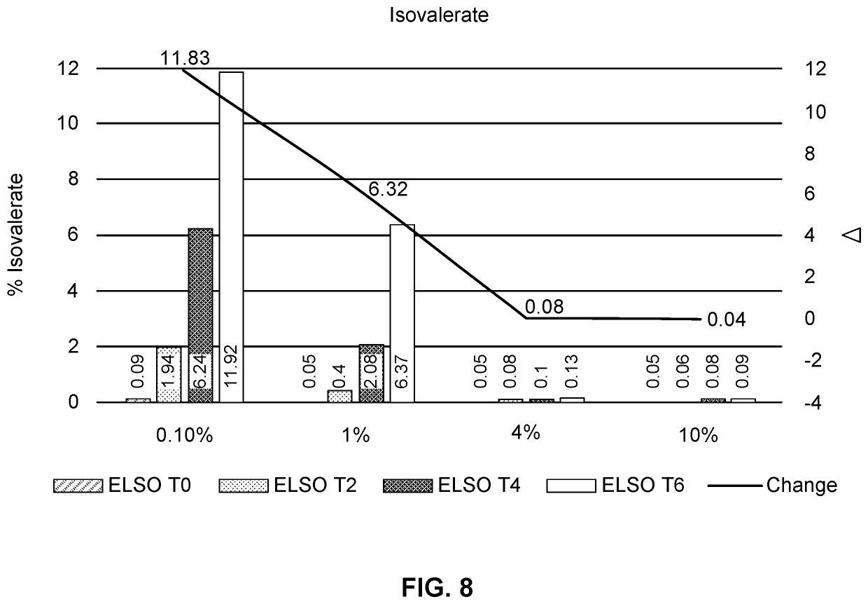

### DETAILED DESCRIPTION

### Definitions

The indefinite articles “a,” “an,” and “the” include plural references unless clearly contradicted or the context clearly dictates otherwise.

The term “alkyl” as used herein by itself or as part of another group refers to a straight-chain or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In some embodiments, the alkyl can be a C₁-C₁₀ alkyl. In other embodiments, the alkyl can be a C₁-C₆ alkyl. In another embodiment, the alkyl can be a C₁-C₄ alkyl. In still further embodiments, the alkyl can be a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, isobutyl, 3-pentyl, hexyl, heptyl, octyl, nonyl, and decyl.

As used herein, the term “optionally substituted alkyl” by itself or as part of another group refers to an alkyl group that is either unsubstituted or substituted with one, two, or three substituents selected from the group consisting of —OH, —NH₂, halogen, —SH, —C≡N, and C₁-C₄ alkoxy.

As used herein the phrase “alkyl nitrite” refers to a chemical compound with an alkyl group attached to a —ONO functional group. In some embodiments, the alkyl is a Cₓ-C_{y} alkyl, such as a C₁-C₁₂ alkyl and the resulting alkyl nitrite is thus referred to as a “C₁-C₁₂ alkyl nitrite.” In other embodiments, the alkyl can be a (C₁-C₇ alkyl group, providing a (C₁-C₇ alkyl nitrite. Non-limiting exemplary alkyl nitrite compounds include isopropyl nitrite, butyl nitrite, sec-butyl nitrite, isobutyl nitrite, tent-butyl nitrite, pentyl nitrite (amyl nitrite), isopentyl nitrite, (isoamyl nitrite), and neo-pentyl nitrite.

As used herein, the term “alkoxy” by itself or as part of another group refers to an alkyl group attached to a terminal oxygen atom. Non-limiting exemplary alkoxy groups include methoxy, ethoxy, tert-butoxy, and the like. In some embodiments, the alkyl is a Cₓ-C_{y} alkyl, such as a C₁-C₆ alkyl and the resulting alkoxy is thus referred to as a “C₁-C₆ alkoxy.” In another embodiment, the alkyl can be a C₁-C₄ alkyl group, providing a C₁-C₄ alkoxy. Non-limiting exemplary alkoxy groups include methoxy, ethoxy, and tert-butoxy.

As used herein, the term “alkylthio” by itself or as part of another group refers to an alkyl group attached to a terminal sulfur atom. In some embodiments, the alkyl is a Cₓ-C_{y} alkyl, such as a C₁-C₄ alkyl group, and the resulting alkylthio group is thus referred to as a “C₁-C₄ alkylthio.” Non-limiting exemplary alkylthio groups include —SCH₃, —SCH₂CH₃ groups, and the like.

As used herein, the term “halogen” by itself or as part of another group refers to —Cl, —F, —Br, or —I.

As used herein, the term “alkenyl” by itself or as part of another group refers to a straight chain or branched alkyl group containing one, two, three, four, five, six, seven, eight, nine, ten, eleven, twelve, thirteen, fourteen, or fifteen double bonds. Non-limiting exemplary alkenyl groups include, but are not limited to, ethenyl, propenyl, isopropenyl, butenyl, sec-butenyl, pentenyl, hexenyl groups, (E,7R,11R)-3,7,11,15-tetramethylhexadec-2-en-1-yl, (6E,10E,14E)-3,7,11,15-tetramethylhexadeca-2,6,10,14-tetraen-1-yl, and (2E,6E,10E,14E,18E,22E)-3,7,11,15,19,23,27-heptamethyloctacosa-2,6,10,14,18,22,26-heptaen-1-yl.

As used herein, the term “optionally substituted alkenyl” by itself or as part of another refers to an alkenyl group that is either unsubstituted or substituted with one, two, or three substituents selected from the group consisting of —OH, —NH₂, halogen, —SH, —C≡N, and C₁-C₄ alkoxy.

As used herein, the term “substituted” refers to independent replacement of one or more (typically 1, 2, 3, 4, or 5) hydrogen atoms on the substituted moiety with substituents independently selected from the group of substituents as specified for a particular group. In general, a non-hydrogen substituent can be any substituent that can be bound to an atom of the given moiety that is specified to be substituted.

As used herein, the term “vitamin K1” refers to the compound 2-methyl-3-[(E,7R,11R)-3,7,11,15-tetramethylhexadec-2-en-1-yl]naphthalene-1,4-dione.

As used herein, the term “menadione” refers to the compound 2-methylnaphthalene-1,4-dione. Menadione is also known in the art, and referred to herein, as vitamin K3.

As used herein, the term “menaquinone” or “menaquinones” refers to known class of vitamin K2 homologues including, but not limited to, MK-1, MK-2, MK-3, MK-4, (2Z)-Menaquinone 4, MK-5, MK-6, MK-7, (2Z)-Menaquinone 7, (6Z)-Menaquinone 7, MK-8, cis-menaquinone 8, MK-9, MK-10, MK-11, MK-12, MK-13, MK-14, and MK-15.

As used herein, “MK-1” refers to the compound 2-methyl-3-(3-methyl-2-buten-1-yl)naphthalene-1,4-dione.

As used herein, “MK-2” refers to the compound 2-methyl-3-[(2E)-3,7-dimethylocta-2,6-dien-1-yl]naphthalene-1,4-dione.

As used herein, “MK-3” refers to the compound 2-methyl-3-[(2E,6E)-3,7,11-trimethyldodeca-2,6,10-trien-1-yl]naphthalene-1,4-dione.

As used herein, “MK-4” refers to the compound 2-methyl-3-[(2E,6E,10E)-3,7,11,15-tetramethylhexadeca-2,6,10,14-tetraen-1-yl]naphthalene-1,4-dione. MK-4 is also commonly referred to in the art as “vitamin K2,” as it is the most common menaquinone found in the human diet. As such, a reference to vitamin K2 in this disclosure refers to MK-4, as opposed to a reference to menaquinone(s), which refers to the class of vitamin K2 derivatives.

As used herein, the term “(2Z)-Menaquinone 4” refers to the compound 2-methyl-3-[(2Z,6E,10E)-3,7,11,15-tetramethylhexadeca-2,6,10,14-tetraen-1-yl]naphthalene-1,4-dione.

As used herein, “MK-5” refers to the compound 2-methyl-3-[(2E,6E,10E,14E)-3,7,11,15,19-pentamethylicosa-2,6,10,14,18-pentaen-1-yl]naphthalene-1,4-dione.

As used herein, “MK-6” refers to the compound 2-methyl-3-[(2E,6E,10E,14E,18E)-3,7,11,15,19,23-hexamethyltetracosa-2,6,10,14,18,22-hexaen-1-yl]naphthalene-1,4-dione.

As used herein, the term “(2Z)-Menaquinone 7” refers to the compound 2-methyl-3-[(2Z,6E,10E,14E,18E,22E)-3,7,11,15,19,23,27-heptamethyloctacosa-2,6,10,14,18,22,26-heptaen-1-yl]naphthalene-1,4-dione.

As used herein, the term “(6Z)-Menaquinone 7” refers to the compound 2-methyl-3-[(2E,6Z,10E,14E,18E,22E)-3,7,11,15,19,23,27-heptamethyloctacosa-2,6,10,14,18,22,26-heptaen-1-yl]naphthalene-1,4-dione.

As used herein, “MK-7” refers to the compound 2-methyl-3-[(2E,6E,10E,14E,18E,22E)-3,7,11,15,19,23,27-heptamethyloctacosa-2,6,10,14,18,22,26-heptaen-1-yl]naphthalene-1,4-dione.

As used herein, “MK-8” refers to the compound 2-methyl-3-[(2E,6E,10E,14E,18E,22E,26E)-3,7,11,15,19,23,27,31-octamethyldotriaconta-2,6,10,14,18,22,26,30-octaen-1-yl]naphthalene-1,4-dione.

As used herein, the term “cis-menaquinone 8” refers to the compound 2-methyl-3-[(2E,6E,10E,14E,18Z,22E,26E)-3,7,11,15,19,23,27,31-octamethyldotriaconta-2,6,10,14,18,22,26,30-octaen-1-yl]naphthalene-1,4-dione.

As used herein, “MK-9” refers to the compound 2-methyl-3-[(2E,6E,10E,14E,18E,22E,26E,30E)-3,7,11,15,19,23,27,31,35-nonamethylhexatriaconta-2,6,10,14,18,22,26,30,34-nonaen-1-yl]naphthalene-1,4-dione.

As used herein, “MK-10” refers to the compound 2-methyl-3-[(2E,6E,10E,14E,18E,22E,26E,30E,34E)-3,7,11,15,19,23,27,31,35,39-decamethyltetraconta-2,6,10,14,18,22,26,30,34,38-decaen-1-yl]naphthalene-1,4-dione.

As used herein, “MK-11” refers to the compound 2-methyl-3-[(2E,6E,10E,14E,18E,22E,26E,30E,34E,38E)-3,7,11,15,19,23,27,31,35,39,43-undecamethyltetratetraconta-2,6,10,14,18,22,26,30,34,38,42-undecaen-1-yl]naphthalene-1,4-dione.

As used herein, “MK-12” refers to the compound 2-methyl-3-[(2E,6E,10E,14E,18E,22E,26E,30E,34E,38E,42E)-3,7,11,15,19,23,27,31,35,39,43,47-dodecamethyloctatetraconta-2,6,10,14,18, 22,26,30,34,38,42,46-dodecaen-1-yl]naphthalene-1,4-dione.

As used herein, “MK-13” refers to the compound 2-methyl-3-[(2E,6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl]naphthalene-1,4-dione.

As used herein, “MK-14” refers to the compound 2-methyl-3-[(2E,6E,10E,14E,18E,22E,26E,30E,34E,38E,42E,46E,50E)-3,7,11,15,19,23,27,31,35,39,43,47,51,55-tetradecamethylhexapentaconta-2,6,10,14,18,22,26,30,34,38,42,46,50,54-tetradecaen-1-yl]naphthalene-1,4-dione.

As used herein, “MK-15” refers to the compound 2-methyl-3-[(2E,6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl]naphthalene-1,4-dione.

As used herein, the abbreviation “ELSO” refers to epoxidized linseed oil.

As used herein, the abbreviation “GC” refers to gas chromatography.

### Description

This disclosure is directed to a composition comprising an alkyl nitrite and an effective amount of at least one compound of Formula I,

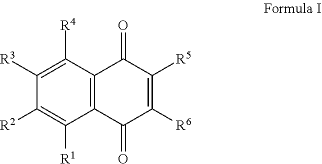

wherein

R¹, R², R³, and R⁴ can each be independently selected from the group consisting of —H, —OH, —NH₂, —SH, halogen, optionally substituted C₁-C₁₂ alkyl, optionally substituted C₁-C₁₂ alkoxy, C₁-C₄ alkylthio, —C≡N, —C(═O )H, —C(═O)OH, —C(═O)O(C₁-C₃ alkyl), and —C(═O)(C₁-C₃ alkyl); and wherein R⁵ and R⁶ can each be independently selected from the group consisting of —H, —OH, —SH, —NH₂, halogen, optionally substituted C₁-C₁₂ alkyl, optionally substituted C₂-C₇₅ alkenyl, C₁-C₄ alkylthio, —C≡N, C₁-C₁₂ alkoxy, —C(═O)H, and —C(═O )(C₁-C₃-alkyl).

In some embodiments, the alkyl nitrite in the composition can be a C₁-C₇ alkyl nitrite. In some embodiments, C₁-C₇ alkyl nitrite can be selected from the group consisting of propyl nitrite, isopropyl nitrite, butyl nitrite, sec-butyl nitrite, isobutyl nitrite, tert-butyl nitrite, amyl nitrite, isoamyl nitrite, and neo-pentyl nitrite. In some embodiments, the alkyl nitrite can be isoamyl nitrite.

In some embodiments, R¹, R², R³, and R⁴ in the at least one compound of Formula I can each be independently selected from the group consisting of —H, —OH, —NH₂, —SH, halogen, optionally substituted C₁-C₁₂ alkyl, and optionally substituted C₁-C₁₂ alkoxy. In some embodiments, each of R¹, R², R³, and R⁴ can be —H.

In certain embodiments, R⁵ and R⁶ can each be independently selected from the group consisting of —H, —OH, —SH, —NH₂, halogen, optionally substituted C₁-C₁₂ alkyl, and optionally substituted C₂-C₇₅ alkenyl.

In some embodiments, R¹, R², R³, and R⁴ can be —H and R⁵ can be an optionally substituted C₁-C₁₂ alkyl. In some embodiments, the optionally substituted C₁-C₁₂ alkyl can be selected from the group consisting of methyl, ethyl, propyl, isopropyl, butyl, sec-butyl, isobutyl, tent-butyl, pentyl, isopentyl, neo-pentyl, (1,1 -dimethyl)-prop-1 -yl, hexyl, heptyl, octyl, isooctyl, nonyl, decyl, cyclopentyl, cyclohexyl, and cycloheptyl. In some embodiments, the optionally substituted C₁-C₁₂ alkyl can be methyl, ethyl, or propyl. In particular embodiments, R¹, R², R³, and R⁴ can be —H and R⁵ can be methyl.

In some embodiments, R¹, R², R³, and R⁴ can be —H and R⁶ can be an optionally substituted C₂-C₇₅ alkenyl or —H. In some embodiments, the optionally substituted C₂-C₇₅ alkenyl can be selected from the group consisting of ethenyl, propenyl, isopropenyl, butenyl, pentenyl, hexenyl, heptenyl, (E,7R,11R)-3,7,11,15-tetramethylhexadec-2-en-1-yl, (Z,7R,11R)-3,7,11,15-tetramethylhexadec-2-en-1-yl, 3-methyl-2-buten-1-yl, (2E)-3,7-dimethylocta-2,6-dien-1-yl, (2E,6E)-3,7,11-trimethyldodeca-2,6,10-trien-1-yl, (2E,6E,10E)-3,7,11,15-tetramethylhexadeca-2,6,10,14-tetraen-1-yl, (2Z,6E,10E)-3,7,11,15-tetramethylhexadeca-2,6,10,14-tetraen-1-yl, (2E,6E,10E,14E)-3,7,11,15,19-pentamethylicosa-2,6,10,14,18-pentaen-1-yl, (2E,6E,10E,14E,18E)-3,7,11,15,19,23-hexamethyltetracosa-2,6,10,14,18,22-hexaen-1-yl, (2Z,6E,10E,14E,18E,22E)-3,7,11,15,19,23,27-heptamethyloctacosa-2,6,10,14,18,22,26-heptaen-1-yl, (2E,6Z,10E,14E,18E,22E)-3,7,11,15,19,23,27-heptamethyloctacosa-2,6,10,14,18,22,26-heptaen-1-yl, (2E,6E,10E,14E,18E,22E)-3,7,11,15,19,23,27-heptamethyloctacosa-2,6,10,14,18,22,26-heptaen-1-yl, (2E,6E,10E,14E,18E,22E,26E)-3,7,11,15,19,23,27,31-octamethyldotriaconta-2,6,10,14,18,22,26,30-octaen-1 -yl, (2E,6E,10E,14E,18Z,22E,26E)-3,7,11,15,19,23,27,31-octamethyldotriaconta-2,6,10,14,18,22,26,30-octaen-1-yl, (2E,6E,10E,14E,18E,22E,26E,30E)-3,7,11,15,19,23,27,31,35-nonamethylhexatriaconta-2,6,10,14,18,22,26,30,34-nonaen-1-yl, (2E,6E,10E,14E,18E,22E,26E,30E,34E)-3,7,11,15,19,23,27,31,35,39-decamethyltetraconta-2,6,10,14,18,22,26,30,34,38-decaen-1-yl, (2E,6E,10E,14E,18E,22E,26E,30E,34E,38E)-3,7,11,15,19,23,27,31,35,39,43-undecamethyltetratetraconta-2,6,10,14,18,22,26,30,34,38,42-undecaen-1-yl, (2E,6E,10E,14E,18E,22E,26E,30E,34E,38E,42E)-3,7,11,15,19,23,27,31,35,39,43,47-dodecamethyloctatetraconta-2,6,10,14,18, 22,26,30,34,38,42,46-dodecaen-1-yl, (2E,6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl, (2E,6E,10E,14E,18E,22E,26E,30E,34E,38E,42E,46E,50E)-2,6,10,14,18,22,26,30,34,38,42,46,50,54-tetradecaen-1-yl, and (2E,6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl.

In some embodiments, R¹, R², R³, and R⁴ can be —H and R⁶ can be an optionally substituted C₂-C₇₅ alkenyl or —H. In some embodiments, the optionally substituted C₂-C₇₅ alkenyl can be selected from the group consisting of ethenyl, propenyl, isopropenyl, butenyl, pentenyl, hexenyl, heptenyl, (E,7R,11R)-3,7,11,15-tetramethylhexadec-2-en-1-yl, and (Z,7R,11R)-3,7,11,15-tetramethylhexadec-2-en-1-yl.

In some embodiments, R¹, R², R³, and R⁴ can be —H and R⁶ can be (E,7R,11R)-3,7,11,15-tetramethylhexadec-2-en-1-yl.

In some embodiments, R¹, R², R³, and R⁴ can be —H; R⁵ can be an optionally substituted C₁-C₁₂ alkyl; and R⁶ can be an optionally substituted C₂-C₇₅ alkenyl or —H. In some embodiments, the optionally substituted C₁-C₁₂ alkyl can be methyl, ethyl, or propyl.

In some embodiments, R¹, R², R³, and R⁴ can be —H; R⁵ can be methyl; and R⁶ can be an optionally substituted C₂-C₇₅ alkenyl or —H. In some embodiments, the optionally substituted C₂-C₇₅ alkenyl can be selected from the group consisting of (E,7R,11R)-3,7,11,15-tetramethylhexadec-2-en-1-yl, 3-methyl-2-buten-1-yl, (2E)-3,7-dimethylocta-2,6-dien-1-yl, (2E,6E)-3,7,11-trimethyldodeca-2,6,10-trien-1-yl, (2E,6E,10E)-3,7,11,15-tetramethylhexadeca-2,6,10,14-tetraen-1-yl, (2E,6E,10E,14E)-3,7,11,15,19-pentamethylicosa-2,6,10,14,18-pentaen-1-yl, (2E,6E,10E,14E,18E)-3,7,11,15,19,23-hexamethyltetracosa-2,6,10,14,18,22-hexaen-1-yl, (2E,6E,10E,14E,18E,22E)-3,7,11,15,19,23,27-heptamethyloctacosa-2,6,10,14,18,22,26-heptaen-1-yl, (2E,6E,10E,14E,18E,22E,26E)-3,7,11,15,19,23,27,31-octamethyldotriaconta-2,6,10,14,18,22,26,30-octaen-1-yl, (2E,6E,10E,14E,18E,22E,26E,30E)-3,7,11,15,19,23,27,31,35-nonamethylhexatriaconta-2,6,10,14,18,22,26,30,34-nonaen-1-yl, (2E,6E,10E,14E,18E,22E,26E,30E,34E)-3,7,11,15,19,23,27,31,35,39-decamethyltetraconta-2,6,10,14,18,22,26,30,34,38-decaen-1-yl, (2E,6E,10E,14E,18E,22E,26E,30E,34E,38E)-3,7,11,15,19,23,27,31,35,39,43-undecamethyltetratetraconta-2,6,10,14,18,22,26,30,34,38,42-undecaen-1-yl, (2E,6E,10E,14E,18E,22E,26E,30E,34E,38E,42E)-3,7,11,15,19,23,27,31,35,39,43,47-dodecamethyloctatetraconta-2,6,10,14,18,22,26,30,34,38,42,46-dodecaen-1-yl, (2E, 6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl, (2E,6E,10E,14E,18E,22E,26E,30E,34E,38E,42E,46E,50E)-3,7,11,15,19,23,27,31,35,39,43,47,51,55-tetradecamethylhexapentaconta-2,6,10,14,18,22,26,30,34,38,42,46,50,54-tetradecaen-1-yl, and (2E, 6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl.

In some embodiments, R², R³, and R⁴ can be —H; R⁵ can be methyl; and R⁶ can be —H, such that the at least one compound of Formula I can be vitamin K3.

In some embodiments, R¹, R², R³, and R⁴ can be —H, R⁵ can be methyl, and R⁶ can be (E,7R,11R)-3,7,11,15-tetramethylhexadec-2-en-1-yl, such that the at least one compound of Formula I can be vitamin K1.

In some embodiments, R¹, R², R³, and R⁴ can be —H; R⁵ can be methyl; and R⁶ can be 3-methyl-2-buten-1-yl, such that the at least one compound of Formula I can be MK-1.

In some embodiments, R¹, R², R³, and R⁴ can be —H; R⁵ can be methyl; and R⁶ can be (2E)-3,7-dimethylocta-2,6-dien-1-yl, such that the at least one compound of Formula I can be MK-2.

In some embodiments, R¹, R², R³, and R⁴ can be —H; R⁵ can be methyl; and R⁶ can be (2E,6E)-3,7,11-trimethyldodeca-2,6,10-trien-1-yl, such that the at least one compound of Formula I can be MK-3.

In some embodiments, R², R³, and R⁴ can be —H; R⁵ can be methyl; and R⁶ can be (2E,6E,10E)-3,7,11,15-tetramethylhexadeca-2,6,10,14-tetraen-1-yl, such that the at least one compound of Formula I can be MK-4.

In some embodiments, R¹, R², R³, and R⁴ can be —H; R⁵ can be methyl; and R⁶ can be (2E,6E,10E,14E)-3,7,11,15,19-pentamethylicosa-2,6,10,14,18-pentaen-1-yl, such that the at least one compound of Formula I can be MK-5.

In some embodiments, R¹, R², R³, and R⁴ can be —H; R⁵ can be methyl; and R⁶ can be (2E,6E,10E,14E,18E)-3,7,11,15,19,23-hexamethyltetracosa-2,6,10,14,18,22-hexaen-1-yl, such that the at least one compound of Formula I can be MK-6.

In some embodiments, R¹, R², R³, and R⁴ can be —H; R⁵ can be methyl; and R⁶ can be (2E,6E,10E,14E,18E,22E)-3,7,11,15,19,23,27-heptamethyloctacosa-2,6,10,14,18,22,26-heptaen-1-yl, such that the at least one compound of Formula I can be MK-7.

In some embodiments, R¹, R², R³, and R⁴ can be —H; R⁵ can be methyl; and R⁶ can be (2E,6E,10E,14E,18E,22E,26E)-3,7,11,15,19,23,27,31-octamethyldotriaconta-2,6,10,14,18,22,26,30-octaen-1-yl, such that the at least one compound of Formula I can be MK-8.

In some embodiments, R¹, R², R³, and R⁴ can be —H; R⁵ can be methyl; and R⁶ can be (2E,6E,10E,14E,18E,22E,26E,30E)-3,7,11,15,19,23,27,31,35-nonamethylhexatriaconta-2,6,10,14,18,22,26,30,34-nonaen-1-yl, such that the at least one compound of Formula I can be MK-9.

In some embodiments, R¹, R², R³, and R⁴ can be —H; R⁵ can be methyl; and R⁶ can be (2E,6E,10E,14E,18E,22E,26E,30E,34E)-3,7,11,15,19,23,27,31,35,39-decamethyltetraconta-2,6,10,14,18,22,26,30,34,38-decaen-1-yl, such that the at least one compound of Formula I can be MK-10.

In some embodiments, R¹, R², R³, and R⁴ can be —H; R⁵ can be methyl; and R⁶ can be (2E,6E,10E,14E,18E,22E,26E,30E,34E,38E)-3,7,11,15,19,23,27,31,35,39,43-undecamethyltetratetraconta-2,6,10,14,18,22,26,30,34,38,42-undecaen-1-yl, such that the at least one compound of Formula I can be MK-11.

In some embodiments, R¹, R², R³, and R⁴ can be —H; R⁵ can be methyl; and R⁶ can be (2E,6E,10E,14E,18E,22E,26E,30E,34E,38E,42E)-3,7,11,15,19,23,27,31,35,39,43,47-dodecamethyloctatetraconta-2,6,10,14,18,22,26,30,34,38,42,46-dodecaen-1-yl, such that the at least one compound of Formula I can be MK-12.

In some embodiments, R¹, R², R³, and R⁴ can be —H; R⁵ can be methyl; and R⁶ can be (2E,6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl, such that the at least one compound of Formula I can be MK-13.

In some embodiments, R¹, R², R³, and R⁴ can be —H; R⁵ can be methyl; and R⁶ can be (2E,6E,10E,14E,18E,22E,26E,30E,34E,38E,42E,46E,50E)-3,7,11,15,19,23,31,35,39,43,47,51,55-tetradecamthylhexapentaconta-2,6,10,14,18,22,26,30,34,38,42,46,50,54-tetradecaen-1-yl, such that the at least one compound of Formula I can be MK-14.

In some embodiments, R¹, R², R³, and R⁴ can be —H; R⁵ can be methyl; and R⁶ can be (2E,6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl, such that the at least one compound of Formula I can be MK-15.

Typically, the at least one compound of Formula I can be present in the composition in an effective amount. In certain embodiments, the effective amount of the at least one compound of Formula I can range from about 0.01% by weight to about 20% by weight of the composition. In certain embodiments, the effective amount of the at least one compound of Formula I can range from about 0.05% by weight to about 20% by weight of the composition. In certain embodiments, the effective amount of the at least one compound of Formula I can range from about 0.1% by weight to about 20% by weight of the composition. In certain embodiments, the effective amount of the at least one compound of Formula I can range from about 1% by weight to about 20% by weight of the composition. In some embodiments, the effective amount of the at least one compound of Formula I can range from about 2% by weight to about 19% by weight of the composition. In some embodiments, the effective amount of the at least one compound of Formula I can range from about 3% by weight to about 18% by weight of the composition. In some embodiments, the effective amount of the at least one compound of Formula I can range from about 4% by weight to about 17% by weight of the composition. In other embodiments, the effective amount of the at least one compound of Formula I can range from about 5% by weight to about 16% by weight of the composition. In other embodiments, the effective amount of the at least one compound of Formula I can range from about 6% by weight to about 15% by weight of the composition. In further embodiments, the effective amount of the at least one compound of Formula I can range from about 7% by weight to about 14% by weight of the composition. In further embodiments, the effective amount of the at least one compound of Formula I can range from about 8% by weight to about 13% by weight of the composition. In further embodiments, the effective amount of the at least one compound of Formula I can range from about 9% by weight to about 12% by weight of the composition. In still further embodiments, the effective amount of the at least one compound of Formula I can range from about 9% by weight to about 11% by weight of the composition. In particular embodiments, the effective amount of the at least one compound of Formula I can be about 10% by weight of the composition, with the remainder of the composition being alkyl nitrite such as isoamyl nitrite.

In certain embodiments, the at least one compound of Formula I present in an effective amount can be a compound wherein R², R³, and R⁴ are each —H. In further embodiments, the at least one compound of Formula I present in an effective amount can be a compound wherein R¹, R², R³, and R⁴ are each —H and wherein R⁵ and R⁶ are each independently selected from the group consisting of —H, —OH, —SH, —NH₂, halogen, optionally substituted C₁-C₁₂ alkyl, and optionally substituted C₂-C₇₅ alkenyl. In still further embodiments, the at least one compound of Formula I present in an effective amount can be a compound wherein R¹, R², R³, and R⁴ are each —H, wherein R⁵ can be an optionally substituted C₁-C₁₂ alkyl, and wherein R⁶ can be an optionally substituted C₂-C₇₅ alkenyl or —H. For example, and in some embodiments, the optionally substituted C₁-C₁₂ alkyl can be selected from the group consisting of methyl, ethyl, propyl, isopropyl, butyl, sec-butyl, isobutyl, tent-butyl, pentyl, isopentyl, neo-pentyl, (1,1-dimethyl)-prop-1-yl, hexyl, heptyl, octyl, isooctyl, nonyl, decyl, cyclopentyl, cyclohexyl, and cycloheptyl. In other embodiments, the optionally substituted C₁-C₁₂ alkyl can be methyl, ethyl, or propyl. In particular embodiments, R², R³, and R⁴ can be —H and R⁵ can be methyl.

In further embodiments, the optionally substituted C₂-C₇₅ alkenyl in the at least one compound of Formula I can be (E,7R,11R)-3,7,11,15-tetramethylhexadec-2-en-1-yl, 3-methyl-2-buten-1-yl, (2E)-3,7-dimethylocta-2,6-dien-1-yl, (2E,6E)-3,7,11-trimethyldodeca-2,6,10-trien-1-yl, (2E,6E,10E)-3,7,11,15-tetramethylhexadeca-2,6,10,14-tetraen-1-yl, (2E,6E,10E,14E)-3,7,11,15,19-pentamethylicosa-2,6,10,14,18-pentaen-1-yl, (2E,6E,10E,14E,18E)-3,7,11,15,19,23-hexamethyltetracosa-2,6,10,14,18,22-hexaen-1-yl, (2E,6E,10E,14E,18E,22E)-3,7,11,15,19,23,27-heptamethyloctacosa-2,6,10,14,18,22,26-heptaen-1-yl, (2E,6E,10E,14E,18E,22E,26E)-3,7,11,15,19,23,27,31-octamethyldotriaconta-2,6,10,14,18,22,26,30-octaen-1-yl, (2E,6E,10E,14E,18E,22E,26E,30E)-3,7,11,15,19,23,27,31,35-nonamethylhexatriaconta-2,6,10,14,18,22,26,30,34-nonaen-1-yl, (2E,6E,10E,14E,18E,22E,26E,30E,34E)-3,7,11,15,19,23,27,31,35,39-decamethyltetraconta-2,6,10,14,18,22,26,30,34,38-decaen-1-yl, (2E,6E,10E,14E,18E,22E,26E,30E,34E,38E)-3,7,11,15,19,23,27,31,35,39,43-undecamethyltetratetraconta-2,6,10,14,18,22,26,30,34,38,42-undecaen-1 -yl, (2E,6E,10E,14E,18E,22E,26E,30E,34E,38E,42E)-3,7,11,15,19,23,27,31,35,39,43,47-dodecamethyloctatetraconta-2,6,10,14,18, 22,26,30,34,38,42,46-dodecaen-1-yl, (2E,6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl, (2E,6E,10E,14E,18E,22E,26E,30E,34E,38E,42E,46E,50E)-3,7,11,15,19,23,27,31,35,39,43,47,51,55-tetradecamethylhexapentaconta-2,6,10,14,18,22,26,30,34,38,42,46,50,54-tetradecaen-1-yl, or (2E,6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl.

In some embodiments, the at least one compound of Formula I present in an effective amount can be at least one of vitamin K1, vitamin K2, or vitamin K3. In some embodiments, the at least one compound of Formula I present in an effective amount can be a combination of any of vitamin K1, vitamin K2, and vitamin K3, such as a combination of vitamin K1 and K2, a combination of vitamin K1 and K3, a combination of vitamin K2 and K3, or a combination of vitamins K1, K2, and K3. In other embodiments, the at least one compound of Formula I can be a menaquinone or combination of menaquinones. In certain embodiments, the at least one compound of Formula I can be selected from the group consisting of MK-1, MK-2, MK-3, MK-4, (2Z)-Menaquinone 4, MK-5, MK-6, MK-7, (2Z)-Menaquinone 7, (6Z)-Menaquinone 7, MK-8, cis-menaquinone 8, MK-9, MK-10, MK-11, MK-12, MK-13, MK-14, MK-15, and combinations thereof.

For example, and in certain embodiments, the combination of menaquinones can be a combination of MK-4 and MK-1, MK-4 and MK-2, MK-4 and MK-3, MK-4 and MK-5, MK-4 and MK-6, MK-4 and MK-7, MK-4 and MK-8, MK-4 and MK-9, MK-4 and MK-10, MK-4 and MK-11, MK-4 and MK-12, MK-4 and MK-13, MK-4 and MK-14, or a combination of MK-4 and MK-15. In other embodiments, the combination of menaquinones can be a combination of MK-4, MK-1, and MK-2; MK-4, MK-3, and MK-5; MK-4, MK-6, and MK-7; MK-4, MK-8, and MK-9; MK-4, MK-10, and MK-11; MK-4, MK-12, and MK-13; or a combination of MK-4, MK-14, and MK-15. In other embodiments, the combination of menaquinones can be a combination of MK-4, MK-1, MK-2, and MK-3; MK-4, MK-5, MK-6, and MK-7; MK-4, MK-8, MK-9, and MK-10; MK-4, MK-11, MK-12, and MK-13; or a combination of MK-4, MK-13, MK-14, and MK-15. In further embodiments, the combination of menaquinones can be a combination of MK-4, MK-1, MK-2, MK-3, and MK-5; MK-4, MK-6, MK-7, MK-8, and MK-9; MK-4, MK-10, MK-11, MK-12, and MK-13; or a combination of MK-4, MK-12, MK-13, MK-14, and MK-15.

In still further embodiments, the combination of menaquinones can be a combination of MK-4, MK-1, MK-2, MK-3, MK-5, and MK-6; MK-4, MK-7, MK-8, MK-9, MK-10, and MK-11; or a combination of MK-4, MK-11, MK-12, MK-13, MK-14, and MK-15. In certain embodiments, the combination of menaquinones can be a combination of MK-4, MK-1, MK-2, MK-3, MK-5, MK-6, and MK-7; MK-4, MK-8, MK-9, MK-10, MK-11, MK-12, and MK-13; or a combination of MK-4, MK-10, MK-11, MK-12, MK-13, MK-14, and MK-15. In some embodiments, the combination of menaquinones can be a combination of MK-4, MK-1, MK-2, MK-3, MK-5, MK-6, MK-7, and MK-8; or a combination of MK-4, MK-9, MK-10, MK-11, MK-12, MK-13, MK-14, and MK-15. In other embodiments, the combination of menaquinones can be a combination of MK-4, MK-1, MK-2, MK-3, MK-5, MK-6, MK-7, MK-8, and MK-9; or a combination of MK-4, MK-8, MK-9, MK-10, MK-11, MK-12, MK-13, MK-14, and MK-15.

In further embodiments, the combination of menaquinones can be a combination of MK-4, MK-1, MK-2, MK-3, MK-5, MK-6, MK-7, MK-8, MK-9, and MK-10; or a combination of MK-4, MK-7, MK-8, MK-9, MK-10, MK-11, MK-12, MK-13, MK-14, and MK-15. In some embodiments, the combination of menaquinones can be a combination of MK-4, MK-1, MK-2, MK-3, MK-5, MK-6, MK-7, MK-8, MK-9, MK-10, and MK-11; or a combination of MK-4, MK-6, MK-7, MK-8, MK-9, MK-10, MK-11, MK-12, MK-13, MK-14, and MK-15. In other embodiments, the combination of menaquinones can be a combination of MK-4, MK-1, MK-2, MK-3, MK-5, MK-6, MK-7, MK-8, MK-9, MK-10, MK-11, and MK-12; or a combination of MK-4, MK-5, MK-6, MK-7, MK-8, MK-9, MK-10, MK-11, MK-12, MK-13, MK-14, and MK-15.

In still further embodiments, the combination of menaquinones can be a combination of MK-4, MK-1, MK-2, MK-3, MK-5, MK-6, MK-7, MK-8, MK-9, MK-10, MK-11, MK-12, and MK-13; or a combination of MK-4, MK-3, MK-5, MK-6, MK-7, MK-8, MK-9, MK-10, MK-11, MK-12, MK-13, MK-14, and MK-15. In certain embodiments, the combination of menaquinones can be a combination of MK-4, MK-1, MK-2, MK-3, MK-5, MK-6, MK-7, MK-8, MK-9, MK-10, MK-11, MK-12, MK-13, and MK-14; or a combination of MK-4, MK-2, MK-3, MK-5, MK-6, MK-7, MK-8, MK-9, MK-10, MK-11, MK-12, MK-13, MK-14, and MK-15. In some embodiments, the combination of menaquinones can be a combination of MK-4, MK-1, MK-2, MK-3, MK-5, MK-6, MK-7, MK-8, MK-9, MK-10, MK-11, MK-12, MK-13, MK-14, and MK-15.

In some embodiments, the composition can comprise from about 0.01% by weight to about 20% by weight of 2-methyl-3-[(E,7R,11R)-3,7,11,15-tetramethylhexadec-2-en-1-yl]naphthalene-1,4-dione (vitamin K1), with the remainder of the composition comprising the alkyl nitrite. In some embodiments, the composition can comprise from about 0.05% by weight to about 20% by weight of vitamin K1, with the remainder of the composition comprising the alkyl nitrite. In some embodiments, the composition can comprise from about 0.1% by weight to about 20% by weight of vitamin K1, with the remainder of the composition comprising the alkyl nitrite. In some embodiments, the composition can comprise from about 1% by weight to about 20% by weight of vitamin K1, with the remainder of the composition comprising the alkyl nitrite. In certain embodiments, the composition can comprise from about 2% by weight to about 19% by weight of vitamin K1, with the remainder of the composition comprising the alkyl nitrite. In certain embodiments, the composition can comprise from about 3% by weight to about 18% by weight of vitamin K1, with the remainder of the composition comprising the alkyl nitrite. In certain embodiments, the composition can comprise from about 4% by weight to about 17% by weight of vitamin K1, with the remainder of the composition comprising the alkyl nitrite. In certain embodiments, the composition can comprise from about 5% by weight to about 16% by weight of vitamin K1, with the remainder of the composition comprising the alkyl nitrite. In other embodiments, the composition can comprise from about 6% by weight to about 15% by weight of vitamin K1, with the remainder of the composition comprising the alkyl nitrite. In other embodiments, the composition can comprise from about 7% by weight to about 14% by weight of vitamin K1, with the remainder of the composition comprising the alkyl nitrite. In other embodiments, the composition can comprise from about 8% by weight to about 13% by weight of vitamin K1, with the remainder of the composition comprising the alkyl nitrite. In further embodiments, the composition can comprise from about 9% by weight to about 12% by weight of vitamin K1, with the remainder of the composition comprising the alkyl nitrite. In further embodiments, the composition can comprise from about 9% by weight to about 11% by weight of vitamin K1, with the remainder of the composition comprising the alkyl nitrite. In still further embodiments, the composition can comprise about 10% by weight of vitamin K1, with the remainder of the composition comprising the alkyl nitrite. In particular embodiments, the alkyl nitrite can be isoamyl nitrite.

### Alkyl Nitrite Stability

The compositions described herein can exhibit surprising stability upon prolonged storage. For example, a composition initially comprising about 90% by weight isoamyl nitrite and about 10% by weight vitamin K1, can have an isoamyl nitrite purity as measured by GC of about 98%, not including any peaks associated with vitamin K1. Surprisingly, after storage at 40° C. for six months, the isoamyl nitrite can have a purity ranging from about 90% to about 98%, from about 93% to about 98%, or from about 95% to about 97%. In certain embodiments, the isoamyl nitrite can have a purity as measured by GC of about 96% after storage at 40° C. for six months. Total impurities related to isoamyl nitrite decomposition can range from about 2% to about 5%, from about 3% to about 5%, or can be about 4%, all as measured by GC. In some embodiments, the impurities can be isoamyl alcohol, isovalerate, or a combination thereof. That said, other impurities can be present and are included in the GC area percentage of “total impurities,” even if those other impurities structures are unknown. A suitable method for measuring isoamyl nitrite purity by GC, including total impurities, isoamyl alcohol, and isovalerate, is described in Example 1 herein.

In certain embodiments, isoamyl alcohol formation in the compositions described herein can be suppressed during storage at 40° C. for six months. For example, in certain embodiments, the amount of isoamyl alcohol produced as a degradant can increase by less than about 2.5 times relative to the amount of isoamyl alcohol present at t=0, i.e. from about 1.3 GC area percent at t=0, to about 3.2 GC area percent at t=6 months at 40° C. This stands in contrast to a composition comprising less than 10% vitamin K1 by weight, wherein the amount of isoamyl alcohol produced over 6 months at 40° C. can range from about 3 to about 5 times the amount of isoamyl alcohol present at t=0, i.e. from about 3.7 GC area percent to about 6 GC area percent.

In some embodiments, isovalerate formation in the compositions described herein can also be suppressed during storage at 40° C. for six months. For example, in certain embodiments, the amount of isovalerate produced as a degradant can increase by less than about 2.5 times relative to the amount of isovalarate present at t=0, i.e. from about 0.05 GC area percent at t=0, to about 0.1 GC area percent at t=6 months at 40° C. This stands in contrast to a composition comprising less than 10% vitamin K1 by weight, wherein the amount of isovalerate produced over 6 months at 40° C. can range from about 130 to about 170 times the amount of isovalerate present at t=0, i.e. from about 6.5 GC area percent to about 12 GC area percent.

The composition described herein can be prepared and packaged in an appropriate container, such as a glass vessel (e.g. a glass ampoule), a stainless steel vessel, an aluminum vessel (e.g. an anodized aluminum vessel), a plastic vessel, or other vessel, any of which can be lined with an inert organic coating, wherein the vessel can have a cap, such as, a screw cap , crimp cap, or other cap known in the art. In certain embodiments, the composition can be packaged in a container suitable for use in an autoinjector or inhaler. In some embodiments, the composition can be packaged in its container under an inert atmosphere, such as nitrogen or argon.

The embodiments discussed herein will be further clarified in the following examples. It should be understood that these examples are not limiting to the embodiments described above.

### EXAMPLES

### Example 1

Sample compositions were prepared by combining isoamyl nitrite with 0.1%, 1%, or 10% (w/w) vitamin K1. Initial samples were withdrawn to analyze the amount of isoamyl nitrite in the composition at t=0 at 25° C. The sample compositions were then sealed and allowed to age for 2, 4, or 6 months at 40° C. Following aging, the compositions were analyzed to determine the purity of isoamyl nitrite in the sample upon storage. Samples were analyzed using a gas chromatograph (GC) with flame ionization detector fitted with a Restek Rtx-BAC1 column (30 m×0.53 mm ID and 3 μm film thickness). The carrier gas was helium having a flow rate of 25 mL/min; the oxidizer was air with a flow rate of 400 mL/min; and the fuel was hydrogen with a flow rate of 40 mL/min. The initial temperature, 40° C., was held for 5 min. The temperature was then raised to a final temperature of 240° C. at a rate of 10° C./min. Table 1 summarizes the results of this study.

TABLE 1

| | | 0.1% by | 1% by | 10% by |
|---|---|---|---|---|
| | | weight | weight | weight |
| | | Vitamin K1 | Vitamin K1 | Vitamin K1 |
| t = 0 | Isoamyl Nitrite | 98.07 | 98.27 | 98.09 |
| (25° C.) | Purity | | | |
| | (GC Area %) | | | |
| | % Total | 1.93 | 1.73 | 1.91 |
| | Impurities | | | |
| | (GC Area %) | | | |
| | % Isoamyl | 1.22 | 1.21 | 1.28 |
| | Alcohol | | | |
| | (GC Area %) | | | |
| | % Isovalerate | 0.07 | 0.05 | 0.05 |
| | (GC Area %) | | | |
| t = 2 | Isoamyl Nitrite | 92.85 | 95.77 | 97.10 |
| Months | Purity | | | |
| (40° C.) | (GC Area %) | | | |
| | % Total | 7.15 | 4.23 | 2.90 |
| | Impurities | | | |
| | (GC Area %) | | | |
| | % Isoamyl | 2.82 | 2.28 | 1.99 |
| | Alcohol | | | |
| | (GC Area %) | | | |
| | % Isovalerate | 2.19 | 0.87 | 0.06 |
| | (GC Area %) | | | |
| t = 4 | Isoamyl Nitrite | 86.06 | 91.18 | 96.76 |
| Months | Purity | | | |
| (40° C.) | (GC Area %) | | | |
| | % Total | 13.94 | 8.82 | 3.24 |
| | Impurities | | | |
| | (GC Area %) | | | |
| | % Isoamyl | 3.86 | 3.37 | 2.28 |
| | Alcohol | | | |
| | (GC Area %) | | | |
| | % Isovalerate | 5.74 | 3.10 | 0.08 |
| | (GC Area %) | | | |
| t = 6 | Isoamyl Nitrite | 75.1 | 84.66 | 96.04 |
| Months | Purity | | | |
| (40° C.) | (GC Area %) | | | |
| | % Total | 24.90 | 15.34 | 3.96 |
| | Impurities | | | |
| | (GC Area %) | | | |
| | % Isoamyl | 5.60 | 4.37 | 3.18 |
| | Alcohol (GC | | | |
| | Area %) | | | |
| | % Isovalerate | 11.78 | 6.84 | 0.11 |
| | (GC Area %) | | | |

### Example 2 (Comparative Example)

Sample compositions were prepared by combining isoamyl nitrite with 0.1%, 1%, 4%, or 10% (w/w) ELSO. Initial samples were withdrawn to analyze the amount of isoamyl nitrite in the composition at t=0 at 25° C. The sample compositions were then sealed and allowed to age for 2, 4, or 6 months at 40° C. Following aging, the compositions were analyzed using GC according to the method described in Example 1 to determine the purity of isoamyl nitrite in the sample upon storage. Table 2 summarizes the results of this study.

TABLE 2

| | | 0.1% by | 1% by | 4% by | 10% by |
|---|---|---|---|---|---|
| | | weight | weight | weight | weight |
| | | ELSO | ELSO | ELSO | ELSO |
| t = 0 | Isoamyl Nitrite | 98.08 | 98.28 | 98.18 | 97.87 |
| (25° C.) | Purity | | | | |
| | (GC Area %) | | | | |
| | % Total | 1.92 | 1.72 | 1.82 | 2.13 |
| | Impurities | | | | |
| | (GC Area %) | | | | |
| | % Isoamyl | 1.20 | 1.22 | 1.32 | 1.64 |
| | Alcohol | | | | |
| | (GC Area %) | | | | |
| | % Isovalerate | 0.09 | 0.05 | 0.05 | 0.05 |
| | (GC Area %) | | | | |
| t = 2 | Isoamyl Nitrite | 93.37 | 97.06 | 97.81 | 97.55 |
| Months | Purity | | | | |
| (40° C.) | (GC Area %) | | | | |
| | % Total | 6.63 | 2.94 | 2.19 | 2.45 |
| | Impurities | | | | |
| | (GC Area %) | | | | |
| | % Isoamyl | 2.68 | 1.83 | 1.59 | 1.82 |
| | Alcohol | | | | |
| | (GC Area %) | | | | |
| | % Isovalerate | 1.94 | 0.40 | 0.08 | 0.06 |
| | (GC Area %) | | | | |
| t = 4 | Isoamyl Nitrite | 84.95 | 92.92 | 97.44 | 97.22 |
| Months | Purity | | | | |
| (40° C.) | (GC Area %) | | | | |
| | % Total | 15.05 | 7.08 | 2.56 | 2.78 |
| | Impurities | | | | |
| | (GC Area %) | | | | |
| | % Isoamyl | 4.11 | 3.07 | 1.88 | 2.06 |
| | Alcohol | | | | |
| | (GC Area %) | | | | |
| | % Isovalerate | 6.24 | 2.08 | 0.10 | 0.08 |
| | (GC Area %) | | | | |
| t = 6 | Isoamyl Nitrite | 74.77 | 84.67 | 97.33 | 96.68 |
| Months | Purity | | | | |
| (40° C.) | (GC Area %) | | | | |
| | % Total | 25.23 | 15.33 | 2.67 | 3.32 |
| | Impurities | | | | |
| | (GC Area %) | | | | |
| | % Isoamyl | 5.64 | 4.62 | 1.80 | 2.35 |
| | Alcohol | | | | |
| | (GC Area %) | | | | |
| | % Isovalerate | 11.92 | 6.37 | 0.13 | 0.09 |
| | (GC Area %) | | | | |

The studies described above show that compounds of Formula I are surprisingly capable of stabilizing alkyl nitrites, and in particular isoamyl nitrite, for up to 6 months at 40° C.

1. A composition comprising an alkyl nitrite and an effective amount of at least one compound of Formula I: 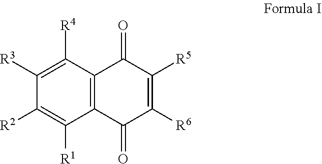
wherein:
R¹, R², R³, and R⁴ are each independently selected from the group consisting of —H, —OH, —NH₂, —SH, halogen, optionally substituted C₁-C₁₂ alkyl, optionally substituted C₁-C₁₂ alkoxy, C₁-C₄ alkylthio, —C≡N, —C(═O)H, —C(═O)OH, —C(═O )O(C₁-C₃ alkyl), and —C(═O )(C₁-C₃ alkyl); further wherein
R⁵ and R⁶ are each independently selected from the group consisting of —H, —OH, —SH, —NH₂, halogen, optionally substituted C₁-C₁₂ alkyl, optionally substituted C₂-C₇₅ alkenyl, C₁-C₄ alkylthio, —C≡N, C₁-C₁₂ alkoxy, —C(═O)H, and —C(═O )(C₁-C₃-alkyl).

2. The composition of claim 1, wherein the alkyl nitrite is a C₁-C₇ alkyl nitrite.

3. The composition of claim 2, wherein the C₁-C₇ alkyl nitrite is selected from the group consisting of propyl nitrite, isopropyl nitrite, butyl nitrite, sec-butyl nitrite, isobutyl nitrite, tent-butyl nitrite, amyl nitrite, isoamyl nitrite, and neo-pentyl nitrite.

4. The composition of claim 3, wherein the alkyl nitrite is isoamyl nitrite.

5. The composition of claim 1, wherein R¹, R², R³, and R⁴ are each —H.

6. The composition of claim 1, wherein R⁵ is an optionally substituted C₁-C₁₂ alkyl and R⁶ is an optionally substituted C₂-C₇₅ alkenyl or —H.

7. The composition of claim 6, wherein the optionally substituted C₁-C₁₂ alkyl is selected from the group consisting of methyl, ethyl, propyl, isopropyl, butyl, sec-butyl, isobutyl, tent-butyl, pentyl, isopentyl, neo-pentyl, (1,1-dimethyl)-prop-1-yl, hexyl, heptyl, octyl, isooctyl, nonyl, decyl, cyclopentyl, cyclohexyl, and cycloheptyl.

8. The composition of claim 6, wherein the optionally substituted C₁-C₁₂ alkyl is methyl.

9. The composition of claim 6, wherein the optionally substituted C₂-C₇₅ alkenyl is selected from the group consisting of ethenyl, propenyl, isopropenyl, butenyl, pentenyl, hexenyl, heptenyl, (E,7R,11R)-3,7,11,15-tetramethylhexadec-2-en-1-yl, (2E)-3,7-dimethylocta-2,6-dien-1-yl, (2E,6E)-3,7,11-trimethyldodeca-2,6,10-trien-1 -yl, (2E,6E,10E)-3,7,11,15-tetramethylhexadeca-2,6,10,14-tetraen-1-yl, (2Z,6E,10E)-3,7,11,15-tetramethylhexadeca-2,6,10,14-tetraen-1-yl, (2E,6E,10E,14E)-3,7,11,15,19-pentamethylicosa-2,6,10,14,18-pentaen-1-yl, (2E,6E,10E,14E,18E)-3,7,11,15,19,23-hexamethyltetracosa-2,6,10,14,18,22-hexaen-1-yl, (2Z,6E,10E,14E,18E,22E)-3,7,11,15,19,23,27-heptamethyloctacosa-2,6,10,14,18,22,26-heptaen-1-yl, (2E,6Z,10E,14E,18E,22E)-3,7,11,15,19,23,27-heptamethyloctacosa-2,6,10,14,18,22,26-heptaen-1-yl, (2E,6E,10E,14E,18E,22E)-3,7,11,15,19,23,27-heptamethyloctacosa-2,6,10,14,18,22,26-heptaen-1-yl, (2E,6E,10E,14E,18E,22E,26E)-3,7,11,15,19,23,27,31-octamethyldotriaconta-2,6,10,14,18,22,26,30-octaen-1 -yl, (2E,6E, 10E,14E,18Z,22E,26E)-3,7,11,15,19,23,27,31-octamethyldotriaconta-2,6,10,14,18,22,26,30-octaen-1-yl, (2E,6E,10E,14E,18E,22E,26E,30E)-3,7,11,15,19,23,27,31,35-nonamethylhexatriaconta-2,6,10,14,18,22,26,30,34-nonaen-1-yl, (2E,6E,10E,14E,18E,22E,26E,30E,34E)-3,7,11,15,19,23,27,31,35,39-decamethyltetraconta-2,6,10,14,18,22,26,30,34,38-decaen-1-yl, (2E,6E,10E,14E,18E,22E,26E,30E,34E,38E)-3,7,11,15,19,23,27,31,35,39,43 -undecamethyltetratetraconta-2,6,10,14,18,22,26,30,34,38,42-undecaen-1-yl, (2E,6E,10E,14E,18E,22E,26E,30E,34E,38E,42E)-3,7,11,15,19,23,27,31,35,39,43,47-dodecamethyloctatetraconta- 2,6,10,14,18, 22,26,30,34,38,42,46-dodecaen-1-yl, (2E,6E,10E,14E,18E,22E,26E,30E,34E,38E,42E,46E)-3,7,11,15,19,23,27,31,35,39,43,47,51-tridecamethyldopentaconta-2,6,10,14,18, 22,26,30,34,38,42,46,50-tridecaen-1-yl, (2E,6E,10E,14E,18E,22E,26E,30E,34E,38E,42E,46E,50E)-3,7,11,15,19,23,27,31,35,39,43,47,51,55-tetradecamethylhexapentaconta -2,6,10,14,18,22,26,30,34,38,42,46,50,54-tetradecaen-1-yl, and (2E,6E,10E,14E,18E,22E,26E,30E,34E,38E,42E,46E,50E,54E)-3,7,11,15,19,23,27,31,35,39,43,47,51,55,59-pentadecamethylhexaconta-2,6,10,14,18,22,26,30,34,38,42,46,50,54,58-pentadecaen-1-yl.

10. The composition of claim 6, wherein the optionally substituted C₂-C₇₅ alkenyl is (E,7R,11R)-3,7,11,15-tetramethylhexadec-2-en-1-yl.

11. The composition of claim 1, wherein R¹, R², R³, and R⁴ are each —H, R⁵ is methyl, and R⁶ is (E,7R,11R)-3,7,11,15-tetramethylhexadec-2-en-1-yl.

12. The composition of claim 1, wherein the effective amount of the at least one compound of Formula I ranges from about 0.1% by weight to about 20% by weight of the composition.

13. The composition of claim 12, wherein the effective amount of the at least one compound of Formula I ranges from about 1% by weight to about 10% by weight of the composition.

14. The composition of claim 13, wherein the effective amount of the at least one compound of Formula I is about 10% by weight of the composition.

15. The composition of claim 13, wherein R¹, R², R³, and R⁴ are each —H, R⁵ is methyl, and R⁶ is (E,7R,11R)-3,7,11,15-tetramethylhexadec-2-en-1-yl.

16. The composition of claim 1, wherein the alkyl nitrite composition is packaged in an ampule under an inert atmosphere.

17. A composition comprising an alkyl nitrite and an effective amount of a vitamin, the vitamin selected from the group consisting of vitamin K1, vitamin K2, vitamin K3, and combinations thereof.

18.-22. (canceled)

23. The composition of claim 17, wherein the effective amount of the vitamin ranges from about 0.1% by weight to about 20% by weight of the composition.

24.-25. (canceled)

26. The composition of claim 17, wherein the alkyl nitrite is a C₁-C₇ alkyl nitrite.

27. The composition of claim 26, wherein the C₁-C₇ alkyl nitrite is selected from the group consisting of propyl nitrite, isopropyl nitrite, butyl nitrite, sec-butyl nitrite, isobutyl nitrite, tent-butyl nitrite, amyl nitrite, isoamyl nitrite, and neo-pentyl nitrite.

28. The composition of claim 27, wherein the alkyl nitrite is isoamyl nitrite.

29. The composition of claim 1, wherein the alkyl nitrite has a purity after storage at 40° C. for six months, ranging from about 90% to about 98%, as measured by gas chromatography (GC).

30. The composition of claim 29, wherein the alkyl nitrite is isoamyl nitrite.

31. The composition of claim 30, wherein the compound of Formula I is vitamin K1.

32. (canceled)
